(19) United States
(12) Patent Application Publication
(10) Pub. No.: US 2006/0252789 A1
(43) Pub. Date: Nov. 9, 2006

(54) Amorphous moxifloxacin hydrochloride

(76) Inventors: Sujay Biswas, West Bengal (IN); Prosenjit Bose, Gurgaon (IN); Yatendra Kumar, Gurgaon (IN)

Correspondence Address: RANBAXY INC., 600 COLLEGE ROAD EAST, SUITE 2100, PRINCETON, NJ, 08540, US

(21) Appl. No.: 10/533,246

(22) PCT Filed: Oct. 30, 2003
(86) PCT No.: PCT/IB03/04845

§ 371 Date: Feb. 16, 2006

(30) Foreign Application Priority Data
Oct. 31, 2002 (IN) 1096/DEL/2002

(51) Int. Cl. A61K 31/4745; C07D 471/02

(52) U.S. Cl. 514/300; 546/123

(57) ABSTRACT

The invention relates to an amorphous form of moxifloxacin hydrochloride and processes for preparing amorphous moxifloxacin hydrochloride. The invention also relates to pharmaceutical compositions that include the amorphous moxifloxacin hydrochloride and use of said compositions for the treatment of bacterial infections.

### FIELD OF THE INVENTION

The field of the invention relates to an amorphous form of moxifloxacin hydrochloride and processes for preparing amorphous moxifloxacin hydrochloride. The invention also relates to pharmaceutical compositions that include the amorphous moxifloxacin hydrochloride and use of said compositions for the treatment of bacterial infections.

### BACKGROUND OF THE INVENTION

Moxifloxacin, a fluoroquinolone, is a broad spectrum antibacterial agent. Moxifloxacin differs from other quinolones in that it has a methoxy function at the 8-position. It is one of the most active quinolones against bacteria which are resistant to penicillins and macrolides.

Chemically, moxifloxacin hydrochloride is 1-cyclopropyl-6-fluoro-1,4-dihydro-8-methoxy-7-[(4aS,7aS)-octahydro-6H-pyrrolo[3,4-b]pyridin-6-yl]-4-oxo-3-quinoline-carboxylic acid hydrochloride salt having structural Formula I.

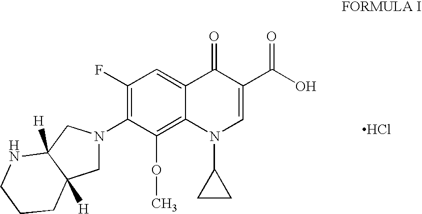

Moxifloxacin and addition products thereof are described in U.S. Pat. No. 4,990,517.

Moxifloxacin and certain specific alkali metal, alkaline earth metal, silver or quanidium salts thereof or a pharmaceutically utilizable hydrate or acid addition salts thereof are disclosed in U.S. Pat. No. 5,607,942.

U.S. Pat. No. 5,849,752 discloses monohydrate of moxifloxacin in prismatic crystal form. However, the inventors are not aware of any disclosure of an amorphous form of moxifloxacin hydrochloride in the prior art. It is known that different morphs of biologically active compounds may have different absorption profile in vivo and consequently different pharmacokinetic profile.

### SUMMARY OF THE INVENTION

In one general aspect there is provided an amorphous form of moxifloxacin hydrochloride.

The amorphous form of moxifloxacin hydrochloride may have the infrared spectrum of FIG. 1 and the X-ray diffraction pattern of FIG. 2.

In another general aspect there is provided a pharmaceutical composition that includes a therapeutically effective amount of an amorphous form of moxifloxacin hydrochloride; and one or more pharmaceutically acceptable carriers, excipients or diluents.

In another general aspect there is provided a process for the preparation of the amorphous form of moxifloxacin hydrochloride. The process includes preparing a solution of moxifloxacin hydrochloride in one or more solvents; and recovering the moxifloxacin hydrochloride in the amorphous form from the solution thereof by the removal of the solvent.

The solvent may be one or more of lower alkanol, ketone, chlorinated solvent, water or mixtures thereof. The lower alkanol may include one or more of primary, secondary and tertiary alcohol having from one to six carbon atoms. The lower alkanol may include one or more of methanol, ethanol, denatured spirit, n-propanol, isopropanol, n-butanol, isobutanol, and t-butanol. In particular, the lower alkanol may include one or more of methanol, ethanol, and denatured spirit.

The ketone may include one or more of acetone, 2-butanone, and 4-methylpentan-2-one. The chlorinated solvent may include one or more of chloroform, dichloromethane and dichloroethane. Removing the solvent may include one or more of distillation, distillation under vacuum, evaporation, spray drying, freeze drying, filtration, filtration under vacuum, decantation and centrifugation.

The moxifloxacin hydrochloride in an amorphous form may be recovered from the solution by spray drying. Alternatively, the moxifloxacin hydrochloride in an amorphous form may be recovered from the solution by freeze-drying. The process may include further forming of the product so obtained into a finished dosage form.

The amorphous form of moxifloxacin hydrochloride can also be recovered from the solution by adding a suitable non-solvent resulting in the precipitation of the amorphous form and removing the solvent there from by filtration, decantation or centrifugation. The non-solvent may be selected from a group of organic solvents in which moxifloxacin hydrochloride is insoluble or poorly soluble or practically insoluble or partially soluble and is known to a person of ordinary skills in the art.

The process may include further drying of the product obtained from the solution.

The process may produce the amorphous form of the moxifloxacin hydrochloride having the infrared spectrum of FIG. 1 and the X-ray diffraction pattern of FIG. 2.

In another general aspect there is provided a method of treating bacterial infections using therapeutically effective amount of the amorphous form of moxifloxacin hydrochloride.

The details of one or more embodiments of the inventions are set forth in the description below. Other features, objects and advantages of the inventions will be apparent from the description and claims.

### DESCRIPTION OF THE DRAWINGS

FIG. 1 is an infrared spectrum in KBr of amorphous form of moxifloxacin hydrochloride.

FIG. 2 is X-ray powder diffraction pattern of amorphous form of moxifloxacin hydrochloride prepared as described herein.

FIG. 3 consists of two infrared spectra, wherein FIG. 3 (A) is the infrared spectrum of anhydrous moxifloxacin hydrochloride and FIG. 3 (B) is the infrared spectrum of monohydrate form of moxifloxacin hydrochloride obtained according to U.S. Pat. No. 5,849,752.

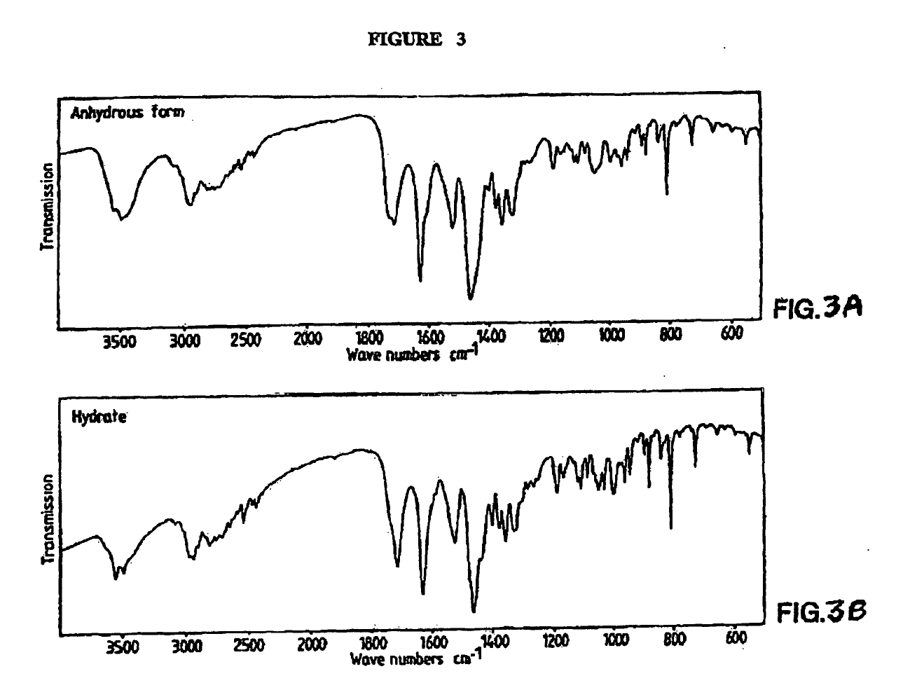

FIG. 4 consists of two X-ray diffractograms, wherein FIG. 4 (A) is the X-ray powder diffraction pattern of anhydrous moxifloxacin hydrochloride and FIG. 4 (B) is X-ray powder diffraction pattern of monohydrate form of moxifloxacin hydrochloride obtained per U.S. Pat. No. 5,849,752.

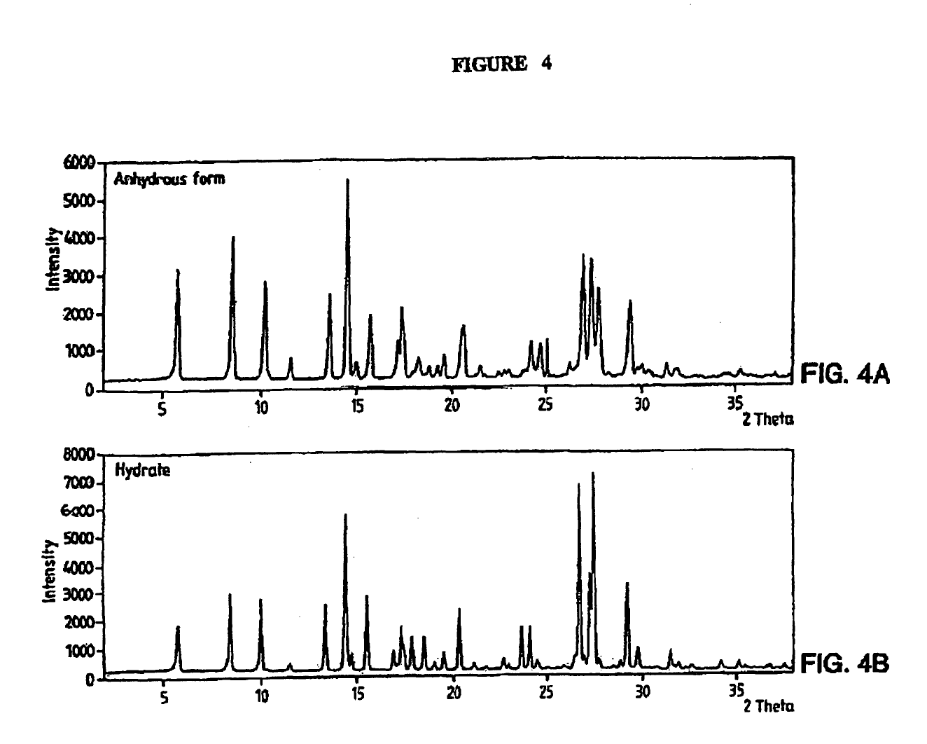

### DETAILED DESCRIPTION OF THE INVENTION

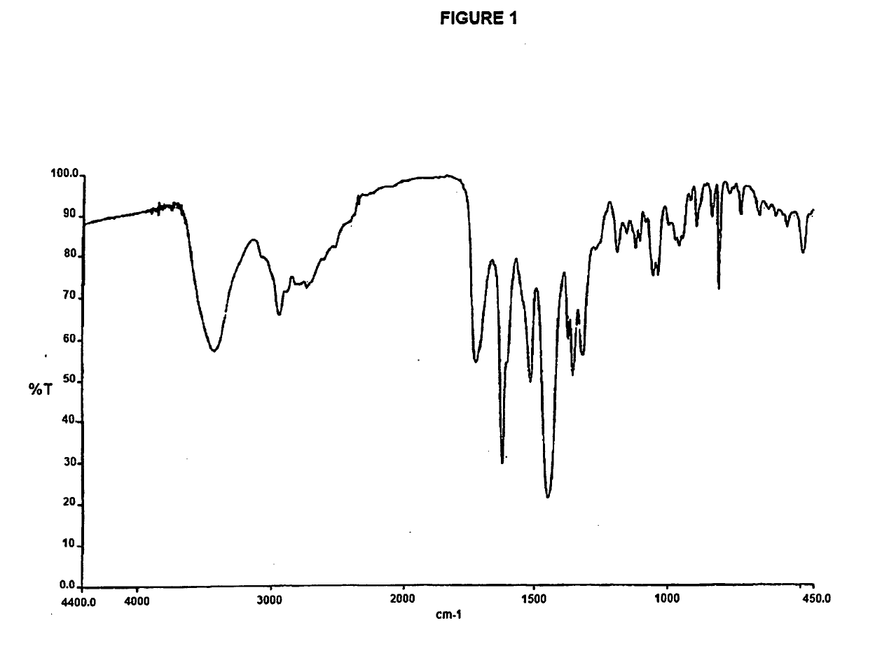

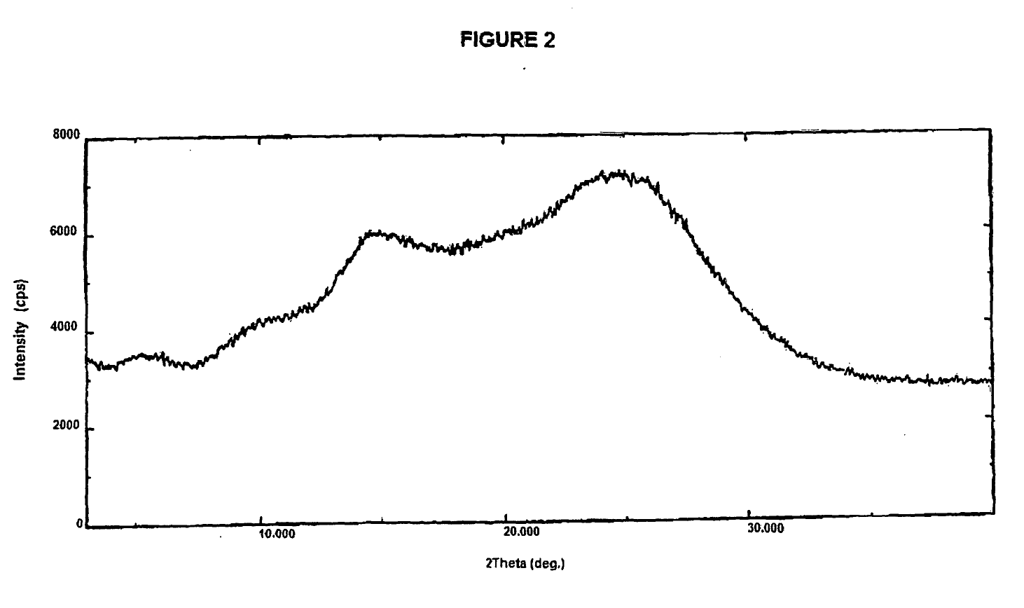

The inventors have found a new form of moxifloxacin, the amorphous form and, in particular, the amorphous moxifloxacin hydrochloride. The new form is characterized by its infrared spectrum and X-ray powder diffraction pattern as shown in FIGS. 1 and 2, -respectively. The inventors also have developed a process for the preparation of the amorphous form of moxifloxacin hydrochloride, by recovering the amorphous moxifloxacin hydrochloride from a solution thereof in a suitable solvent by spray drying. The inventors also have developed pharmaceutical compositions that contain the amorphous form of the moxifloxacin hydrochloride, in admixture with one or more solid or liquid pharmaceutical diluents, carriers, and/or excipients. These pharmaceutical compositions may be used for the treatment of bacterial infection.

In general, the solution of moxifloxacin hydrochloride may be obtained by dissolving a crystalline moxifloxacin hydrochloride in a suitable solvent. Alternatively, such a solution may be obtained directly from a reaction in which moxifloxacin hydrochloride is formed. The solvent may be removed from the solution by a technique which includes, for example, distillation, distillation under vacuum, evaporation, spray drying, freeze drying, filtration, decantation, and centrifugation.

In one aspect, moxifloxacin hydrochloride in amorphous form is recovered from the solution using a spray drying technique. A Mini-Spray Dryer (Model: Buchi 190, Switzerland) can be used. The Buchi 190 Mini-Spray Dryer operates on the principle of nozzle spraying in a parallel flow, i.e., the sprayed product and the drying gas flow in the same direction. The drying gas can be air or inert gases such as nitrogen, argon and carbon dioxide.

In another aspect, moxifloxacin hydrochloride in amorphous form can be recovered from the solution using a freeze drying technique. A freeze dryer (Model: Virtis Genesis SQ Freeze Dryer) can be used in this technique. The Virtis Genesis SQ Freeze Dryer operates on the principle of lyophilization, i.e., a process of stabilizing initially wet materials (aqueous solution or suspensions) by freezing them, then subliming the ice while simultaneously desorbing some of the bound moisture (primary drying). Following removal of the ice, desorption may be continued (secondary drying). This process may be carried out under vacuum.

The term “suitable solvent” includes any solvent or solvent mixture in which moxifloxacin hydrochloride, is soluble, including, for example, lower alkanol, ketones, chlorinated solvents, water and mixtures thereof. Examples of alkanol include those primary, secondary and tertiary alcohols having from one to six carbon atoms. Suitable lower alkanol solvents include methanol, ethanol, denatured spirit, n-propanol, isopropanol, n-butanol, isobutanol and t-butanol. Examples of ketones include solvents such as acetone, 2-butanone, and 4-methylpentan-2-one. A suitable chlorinated solvent includes one or more of dichloromethane, dichloroethane and chloroform. Mixtures of all of these solvents are also contemplated.

If crystalline moxifloxacin hydrochloride is used as a starting material it may be in the form of any of the various polymorphic forms known in the prior art including solvates, hydrates, anhydrous or any other polymorphic forms of moxifloxacin hydrochloride. A solution of moxifloxacin hydrochloride obtained in situ during the preparation process may be used as such for spray drying.

The spray drying may be accomplished using a spray dryer which operates on the principle of nozzle spraying in a parallel flow, i.e., the sprayed product and the drying gas flow in the same direction. The drying gas can be air or one or more inert gases such as nitrogen, argon, and carbon dioxide. Moreover, the product obtained may be further or additionally dried to achieve the desired moisture values. For example, the product may be further or additionally dried in a tray drier, dried under vacuum and/or in a Fluid Bed Dryer.

The resulting amorphous form of moxifloxacin hydrochloride may be formulated into ordinary dosage forms such as, for example, tablets, capsules, pills, solutions, etc. In these cases, the medicaments can be prepared by conventional methods with conventional pharmaceutical excipients.

The compositions include dosage forms suitable for oral, buccal, rectal, and parenteral (including subcutaneous, intramuscular, and ophthalmic) administration. The oral dosage forms may include solid dosage forms, like powder, tablets, capsules, suppositories, sachets, troches and lozenges as well as liquid suspensions, emulsions, pastes and elixirs. Parenteral dosage forms may include intravenous infusions, sterile solutions for intramuscular, subcutaneous or intravenous administration, dry powders to be reconstituted with sterile water for parenteral administration, and the like.

Further, the amorphous moxifloxacin hydrochloride dosage forms described herein can be used in a method for treatment of bacterial infection. The method of treatment includes administering to a mammal in need of treatment a dosage form that includes a therapeutically effective amount of the amorphous form of moxifloxacin hydrochloride.

The present invention is further illustrated by the following example which is provided merely to be exemplary of the invention and is not intended to limit the scope of the invention. Although the example is directed to amorphous form of moxifloxacin hydrochloride, the principles described in this example can be applied to other salts of amorphous moxifloxacin.

Preparation of Amorphous Form of Moxifloxacin Hydrochloride

### EXAMPLE

A suspension was made from crystalline moxifloxacin hydrochloride (20 g) in methanol (600 ml) at ambient temperature. The resulting solution was slowly heated to 40-42° C. for 30 minutes to get a clear solution which was subjected to spray drying in a Mini Spray Dryer (Model Buchi—190) at a temperature of 67-68° C. using nitrogen gas. The moxifloxacin hydrochloride in an amorphous form was collected. It was further dried at 50-55° C. for 10 hours under vacuum to yield amorphous moxifloxacin hydrochloride.

X-ray powder diffraction pattern (FIG. 2) showed a plain halo, which demonstrates the amorphous-nature of the product.

Infrared spectrum in KBr (FIG. 1) is different than one obtained for crystalline form of moxifloxacin hydrochloride.

While the present invention has been described in terms of its specific embodiments, certain modifications and equivalents will be apparent to those skilled in the art and are intended to be included within the scope of the present invention.

We claim:

1. An amorphous form of moxifloxacin hydrochloride.

2. The amorphous form of moxifloxacin hydrochloride of claim 1, wherein the moxifloxacin hydrochloride has the infrared spectrum of FIG. 1.

3. The amorphous form of moxifloxacin hydrochloride of claim 1, wherein the moxifloxacin hydrochloride has the X-ray diffraction pattern of FIG. 2.

4. A pharmaceutical composition comprising:
a therapeutically effective amount of an amorphous form of moxifloxacin hydrochloride; and one or more pharmaceutically acceptable carriers, excipients or diluents.

5. The pharmaceutical composition of claim 1, wherein the moxifloxacin hydrochloride has the infrared spectrum of FIG. 1.

6. The pharmaceutical composition of claim 1, wherein the moxifloxacin hydrochloride has the X-ray diffraction pattern of FIG. 2.

7. A process for the preparation of the amorphous form of moxifloxacin hydrochloride, the process comprising:
preparing a solution of moxifloxacin hydrochloride in one or more solvents; and
recovering the moxifloxacin hydrochloride in the amorphous form from the solution thereof by the removal of the solvent.

8. The process of claim 7, wherein the solvent comprises one or more of lower alkanol, ketone, chlorinated solvent, or mixtures thereof.

9. The process of claim 8, wherein the lower alkanol comprises one or more of primary, secondary and tertiary alcohol having from one to six carbon atoms.

10. The process of claim 8, wherein the lower alkanol comprises one or more of methanol, ethanol, denatured spirit, n-propanol, isopropanol, n-butanol, isobutanol, and t-butanol.

11. The process of claim 8, wherein the lower alkanol comprises one or more of methanol, ethanol, and denatured spirit.

12. The process of claim 8, wherein the ketone comprises one or more of acetone, 2-butanone, and 4-methylpentan-2-one.

13. The process of claim 8, wherein the chlorinated solvent comprises one or more of chloroform, dichloromethane, and dichloroethane.

14. The process of claim 7, wherein removing the solvent comprises one or more of distillation, distillation under vacuum, evaporation, spray drying, freeze drying, filtration, decantation, and centrifugation.

15. The process of claim 7, wherein the moxifloxacin hydrochloride in an amorphous form is recovered from the solution by spray drying.

16. The process of claim 7, wherein the moxifloxacin hydrochloride in an amorphous form is recovered from the solution by freeze-drying.

17. The process of claim 7, wherein the moxifloxacin hydrochloride in an amorphous form is recovered from the solution by filtration.

18. The process of claim 7, further comprising additional drying of the product obtained.

19. The process of claim 7, further comprising forming the product obtained into a finished dosage form.

20. The process of claim 7, wherein the moxifloxacin hydrochloride has the infrared spectrum of FIG. 1.

21. The process of claim 7, wherein the moxifloxacin hydrochloride has the X-ray diffraction pattern of FIG. 2.
